(19) United States
(12) Patent Application Publication
(10) Pub. No.: US 2011/0160485 A1
(43) Pub. Date: Jun. 30, 2011

(54) METHOD FOR THE PRODUCTION OF MIXED OXIDES CONTAINING COPPER AND CHROMIUM

(76) Inventors: Hans-Jörg Wölk, Rosenheim (DE); Alfred Hagemeyer, Bad Aibling (DE); Frank Grossmann, Munchen (DE)

(21) Appl. No.: 12/994,909

(22) PCT Filed: May 29, 2009
(86) PCT No.: PCT/EP2009/003880

§ 371 Date: Feb. 24, 2011

(30) Foreign Application Priority Data
May 30, 2008 (DE) 10 2008 026 208.0

(51) Int. Cl. B01J 23/86; C01G 37/02; C07C 47/00; C07C 209/32; C07C 29/149; B82Y 30/00

(52) U.S. Cl. 564/1; 423/595; 423/596; 502/318; 568/420; 568/700; 428/402; 977/773

(57) ABSTRACT

The present invention relates to a method for producing a nanocrystalline mixed oxide material containing copper and chromium as well as the mixed oxide material containing copper and chromium produced by the method according to the invention and its use as catalyst, in particular for dehydrogenating alcohols, for hydrogenation reactions, for reducing nitrocompounds, for hydrogenating carboxylic acids and for hydrogenating free fatty acids to fatty alcohols.

The invention relates to a method for producing a nanocrystalline mixed oxide containing copper and chromium in a pulsation reactor as well as the use of the produced nanocrystalline mixed oxide containing copper and chromium as catalyst.

Copper chromite is known in the state of the art as a catalyst for dehydrogenating alcohol and for simple hydrogenation reactions, for example for reducing nitrocompounds or for hydrogenating carboxylic acids. In particular the hydrogenation of free fatty acids to fatty alcohols (see Ullmanns Encyklopädie der technischen Chemie, 3^{rd} edition, volume 11, pages 427-445), which can be carried out in the presence of copper chromite catalysts, is of technical interest.

According to a method developed by Adkins (see inter alia J. Schulz et al. in: Zeitschrift fur anorganische and allgemeine Chemie, Volume 346 (1966), pp. 66-75) copper chromite catalysts are usually obtained via intermediately formed copper ammonium hydroxy chromate which is broken down by calcining, i.e. thermally, into copper oxide and copper chromite. The intermediately formed copper ammonium hydroxy chromate is obtained by precipitate formation from ammonium chromate and copper(II) salts. The copper(II) oxide formed in the process is then washed out by acid treatment, e.g. with glacial acetic acid. However, the acid treatment has a disadvantageous effect on the catalytic activity of the copper chromite.

A method for producing an acid-resistant copper chromite spinel catalyst for the direct fixed-bed hydrogenation of fatty acids is described in the unexamined German application DE-OS 3706658. For this, copper(II) chromite is produced, via the stage of intermediately formed copper ammonium hydroxy chromate, in the presence of colloidal silica gel in per se known manner, and annealed at temperatures around 750° C. over a period of at least 12 hours. The thus-obtained copper(II) chromite spinel catalyst contains barium and manganese salts as so-called energy promoters. However, the high annealing temperature leads to the formation of unwanted by-products, as a result of which the activity of a copper chromite catalyst produced in this way is reduced.

An improvement in the catalyst activity of copper chromite catalysts by adding manganese and/or barium salts in quantities of up to 10 wt.-% is also known from the European patent specifications EP-PS 69 339, EP-PS 1 276 722 and EP-PS 0023 699.

Despite these technical advancements, copper chromite catalysts according to the state of the art unfortunately still have too low an activity.

The object of the present invention was thus to provide a copper chromite catalyst which has an increased activity.

The object is achieved by a method for producing a nanocrystalline mixed oxide containing copper and chromium, comprising the steps of
a) the introduction of a solution, suspension or slurry, containing starting compounds containing copper and containing chromium or a starting compound containing copper and chromium, into a reaction chamber by means of a carrier fluid,
b) a thermal treatment of the starting compounds containing copper and containing chromium or of the starting compound containing copper and chromium in a treatment zone by means of a pulsating flow at a temperature of from 200 to 700° C.,
c) the formation of a nanocrystalline mixed oxide containing copper and chromium,
d) the discharge of the nanocrystalline mixed oxide containing copper and chromium obtained in steps b) and c) from the reactor.

The starting compounds containing copper and containing chromium or the starting compound containing copper and chromium are or is preferably selected from the group comprising complex chromate compounds, copper amine carbonate, chromic acids and copper hydroxy carbonate. The complex chromate compound is preferably a copper chromate.

It is further preferred that a starting compound containing barium and/or containing manganese is also used as additional starting compound. Barium and manganese are known as promoters. The starting compound containing barium is preferably a barium chromate, particularly preferably a complex barium chromate and the starting compound containing manganese is preferably a manganese chromate, particularly preferably a complex manganese chromate.

Thus within the meaning of the general description of the invention the term “containing copper and chromium” in relation to mixed oxides also covers the further preferred elements, such as barium and/or manganese, without this needing to be mentioned specifically. Thus, by a mixed oxide containing copper and chromium within the meaning of this invention is also meant a mixed oxide containing copper, chromium, barium and/or manganese.

The term “mixed oxide” is mainly understood to refer to both homeotypic and heterotypic mixed oxide according to the definition of mixed crystals in: Hollemann-Wiberg, Lehrbuch der Anorganischen Chemie, 102^{nd} edition, de Gruyter Berlin 2007, p. 130ff, i.e. the, metal ions occupy crystal lattice sites or are present as mixed phases (see p. 1404ff op. cit.). In less preferred configurations the mixed oxide is also present as a physical mixture of the individual oxides or of the individual mixed oxides.

It was surprisingly found that the method can be carried out at relatively low temperatures of from 200 to 700° C., particularly preferably of from 230 to 680° C., particularly preferably of from 250 to 650° C. Hitherto, preferred temperatures of more than 700° C., indeed up to 1400° C., were known in the state of the art. Quite particularly surprisingly, it was also found that the crystallization process of the mixed oxide containing copper and chromium can be controlled in a targeted manner by the method according to the invention, in particular the size of the crystallites and the pore-size distribution of the corresponding mixed oxide containing copper and chromium. This can moreover be further advantageously influenced by the residence time in the flame or by the reactor temperature. The mixed oxide particles containing copper and chromium that form are prevented from agglomerating by the pulsating thermal treatment. Typically, the nanocrystalline particles are immediately transferred through the stream of hot gas into a colder zone, where the primary crystallites containing copper and chromium, some with diameters of less than 150 nm, preferably 4 to 150 nm, are obtained.

In the case of the thus-obtainable mixed oxide crystallites containing copper and chromium, this leads to clearly increased BET surface areas of >40 m²/g, preferably >150 m/g, particularly preferably >200 m²/g and in particular up to 300 m²/g. The BET surface area is determined according to DIN 66132 (using the Brunauer, Emmett and Teller method). The increased BET surface area leads to a dramatically increased catalytic activity of the nanocrystalline mixed oxide containing copper and chromium.

In the method according to the invention, suspensions can be calcined within a very short period, typically within a few milliseconds, at comparatively lower temperatures than are usual with methods of the state of the art, without additional filtration and/or drying steps or without the addition of additional solvents. The mixed oxide crystallites containing copper and chromium that form have very high BET surface areas. A mixed oxide catalyst containing copper and chromium with increased reactivity, improved rate of conversion and improved selectivity compared with the catalysts of the state of the art is thus accessible.

The nearly identical residence time of every mixed oxide particle containing copper and chromium in the homogeneous temperature field created by the method results in an extremely homogeneous end product with narrow monomodal particle distribution. A device for carrying out the method according to the invention in the production of such monomodal nanocrystalline metal oxide powders is known for example from DE 101 09 892 A1. Unlike the device described there and the method disclosed there, the present method does not, however, require an upstream evaporation step in which the starting material, i.e. the nickel starting compound, is heated to an evaporation temperature.

The starting compound containing copper and chromium (and optionally containing barium and/or manganese) from which the mixed oxide materials containing copper and chromium according to the invention are produced are inserted directly via a carrier fluid, in particular a carrier gas, preferably an inert carrier gas, such as for example nitrogen, etc., into so-called reaction chambers, i.e. into the combustion chamber. Attached exhaust-side to the reaction chamber is a resonance tube with a flow cross-section which is clearly reduced compared with the reaction chamber. The floor of the combustion chamber is equipped with several valves for the entry of the combustion air into the combustion chamber. The aerodynamic valves are fluidically and acoustically matched to the combustion chamber and the resonance tube geometry such that the pressure waves, created in the combustion chamber, of the homogeneous “flameless” temperature field spread pulsating predominantly in the resonance tube. A so-called Helmholtz resonator forms with pulsating flow with a pulsation frequency of between 3 and 150 Hz, preferably 5 to 50 Hz.

Material is typically fed into the reaction chamber either with an injector or with a suitable two-component or three-component nozzle or in a Schenk dispenser.

The starting compounds are preferably introduced into the reaction chamber in atomized form, with the result that a fine distribution in the region of the treatment zones is guaranteed.

The starting compounds can be introduced either jointly or separately into the reaction chamber. Introduction can advantageously be via a two-component or three-component nozzle, wherein the starting compounds are either sprayed direct and the mixture thus forms in the reaction chamber, or by producing a mixture first and then spraying this.

The starting compounds containing copper and chromium (and optionally the further starting compounds) are preferably obtained via a wet-chemical method. A preferred starting compound containing copper and chromium is for example a complex copper-barium-ammonium chromate.

The complex copper-barium-ammonium chromate is produced for example by using a mixture of barium nitrate and distilled water to make a solution to which copper nitrate trihydrate is additionally added. A solution of ammonium chromate in aqueous ammonia is then added to this solution. The preferably hot solution of the nitrates is stirred while the ammonium chromate solution is added. Stirring is continued, wherein a reddish-brown precipitate of copper-barium-ammonium chromate forms.

The reaction can also take place without adding barium. However, it is known that barium as catalyst component displays a protective effect against sulphate poisoning and also has a stabilizing effect on the catalyst vis-à-vis reduction. Manganese can also be used as promoter and stabilizer, or barium and manganese in combination.

This copper-(barium and/or manganese)-ammonium chromate precipitate formed according to the method described above is subjected to a thermal treatment in the pulsation reactor, preferably after a cleaning. It can also be loaded into the pulsation reactor without prior cleaning.

After the thermal treatment, the nanocrystalline mixed oxides containing copper and chromium (with or without barium and/or manganese) that form are immediately transferred into a colder zone of the reaction chamber, if possible by means of the carrier fluid, with the result that they can be separated and discharged in the colder zone. The yield of the method according to the invention is almost 100%, as all of the product that forms can be discharged from the reactor.

Typically, the method is carried out at a pressure in the range of from normal pressure to 40 bar.

A subject of the invention is furthermore the nanocrystalline mixed oxide material containing copper and chromium that can be obtained by the method according to the invention. It was found that the thus-obtainable nanocrystalline mixed oxide material containing copper and chromium preferably has a crystallite size in the range of from 4 nm to 150 nm, preferably from 10 nm to 120 nm, quite particularly preferably 10 to 100 nm, which, as already stated above, can preferably be set by the pulsation of the thermal treatment. The particle size can be determined by XRD and/or TEM.

The mixed oxide particles containing copper and chromium obtained by the method according to the invention have a BET surface area of preferably >40 m²/g, particularly preferably >100 m²/g, particularly preferably >150 m²/g and in particular of up to 300 m²/g.

The mixed oxide containing copper and chromium preferably further contains a barium and/or manganese, preferably barium chromate and/or manganese chromate in the form of a mixed crystal, a mixed phase or in a less preferred embodiment as a physical mixture.

The mixed oxide material containing copper and chromium according to the invention, preferably copper chromite, is exceptionally suitable for use as catalyst or as catalyst precursor, in particular as catalyst for dehydrogenating alcohols, for hydrogenation reactions, for reducing nitrocompounds, for hydrogenating carboxylic acids and for hydrogenating free fatty acids or fatty acid esters to fatty alcohols.

The catalyst can be formed as coated catalyst or as solid catalyst, thus be present in the form of a bulk catalyst, shell catalyst or as extrudate. In general catalysts can be divided into solid catalysts and coated catalysts. While solid catalysts consist of more than 50% catalytically active material, coated catalysts consist of a catalyst support body, wherein the surface of the catalyst support body is provided with a coating. The coating is in most cases applied to the catalyst support by means of a so-called washcoat suspension, i.e. a slurry in a fluid medium. The applied washcoat suspension is usually then dried and calcined. The coating can then be impregnated with a further catalytically active component, wherein the active components can also be dissolved in the washcoat suspension or have been applied beforehand to the metal oxide particles. The advantage of coated catalysts is the simple production, which is associated with a small outlay on process engineering.

A binder is preferably added when producing extrudates. Peptized aluminium oxide hydrates for example can be used as binders for producing extrudates. These include for example boehmite, which can be obtained under the trade name “Dural SCF” (producer: SASOL). These compounds provide excellent support for shaping. This is based on their ability to peptize in the presence of monovalent acids.

The extrudates can have any geometric shapes, for example bars, hollow cylinders, monoliths and the like.

The catalyst can also be used in the form of powder.

The invention will now be explained in more detail with reference to the following embodiment examples, which are not to be regarded as limitative. The device used largely corresponds, as already mentioned above, to the device described in DE 101 09 892 A1, with the difference that the device used for carrying out the method according to the invention had no preliminary evaporator stage.

### EMBODIMENT EXAMPLES

### Example 1

A mixture of 26.0 g (0.1 mol) barium nitrate and 800 ml distilled water is heated to 70° C. Once a complete solution has formed, 218 g (0.9 mol) copper nitrate trihydrate is added and the mixture stirred at 70° C. until a clear solution forms.

A solution of ammonium chromate is produced by dissolving 126 g (0.5 mol) ammonium dichromate in 600 ml distilled water and 150 ml 28% aqueous ammonia is added to this. The hot solution of the nitrates is stirred while the ammonium chromate solution is added in a thin jet. Stirring is continued for a few minutes, wherein a reddish-brown precipitate of copper, barium, ammonium chromate forms which is filtered off via a 16-cm Büchner funnel and dried at 110° C.

The produced copper chromate compound is converted to a suspension in distilled water. The suspension is sprayed into the thermal unit via a two-component nozzle with a feed quantity of 14 kg/hr.

The following BET surface areas were obtained at different temperatures:

300° C.: 47 m²/g

350° C.: 56 m²/g

400° C.: 72 m²/g

450° C.: 80 m²/g

Barium was used in this example since, as catalyst component, it displays a protective effect against sulphate poisoning and also has a stabilizing effect on the catalyst vis-à-vis reduction.

### Example 2

This example is carried out analogously to Example 1, but was carried out without using barium. The barium nitrate was thus omitted and 242 g (1 mol) copper nitrate added instead. All other details correspond to Example 1.

The produced copper chromate compound is converted to a suspension with distilled water. The suspension is sprayed into the thermal unit via a two-component nozzle with a feed quantity of 14 kg/hr.

The BET surface areas obtained lay in the same range as in Example 1.

### Example 3

Example 3 was carried out analogously to Example 1, but the solutions were sprayed without prior mixing with the help of a three-component nozzle.

The BET surface areas obtained were comparable to those from Example 1.

### Example 4

Example 4 was carried out analogously to Example 1, but 0.1 mol manganese nitrate was used instead of barium nitrate.

The BET surface areas obtained were comparable to those from Example 1.

### Example 5

Example 5 was carried out analogously to Example 1, but a mixture of 0.05 mol barium nitrate and 0.05 mol manganese nitrate was used.

The BET surface areas obtained were comparable to those from Example 1.

1. Method for producing nanocrystalline mixed oxides containing copper and chromium, comprising the steps of
a) the introduction of a solution, suspension or slurry, containing starting compounds containing copper and containing chromium or a starting compound containing copper and chromium, into a reaction chamber by means of a carrier fluid,
b) a thermal treatment of the starting compounds containing copper and containing chromium or of the starting compound containing copper and chromium in a treatment zone by means of a pulsating flow at a temperature of from 200 to 700° C.,
c) the formation of a nanocrystalline mixed oxide containing copper and chromium,
d) the discharge of the nanocrystalline mixed oxide containing copper and chromium obtained in steps b) and c) from the reactor.

2. Method according to claim 1, characterized in that the starting compounds containing copper and containing chromium or the starting compound containing copper and chromium are selected from the group comprising complex chromate compounds, copper amine carbonate, chromic acids and copper hydroxy carbonate.

3. Method according to claim 2, characterized in that the complex chromate compound is a copper chromate.

4. Method according to claim 1, characterized in that additionally a starting compound containing barium and/or containing manganese is also used.

5. Method according to claim 4, characterized in that the starting compound containing barium is a complex barium chromate.

6. Method according to claim 4, characterized in that the starting compound containing manganese is a complex manganese chromate.

7. Mixed oxide containing copper and chromium that can be obtained according to a method according to claim 1.

8. Mixed oxide containing copper and chromium according to claim 7, characterized in that the mixed oxide containing copper and chromium is amorphous.

9. Mixed oxide containing copper and chromium according to claim 7, characterized in that it has a BET surface area of from 40 to 300 m²/g.

10. Mixed oxide containing copper and chromium according to claim 7, characterized by a particle size of the primary crystallites of from 4 to 150 nm.

11. Mixed oxide containing copper and chromium according to claim 7, characterized in that it further comprises barium and/or manganese.

12. Mixed oxide containing copper and chromium according to claim 7, characterized in that the barium is present as barium chromate.

13. Mixed oxide containing copper and chromium according to claim 7, characterized in that the manganese is present as manganese chromate.

14. In a method for catalyzing a reaction, the improvement comprising using as a catalyst the mixed oxide containing copper and chromium according to claim 7.

15. A method for dehydrogenating alcohols, for hydrogenation reactions, for reducing nitrocompounds, for hydrogenating carboxylic acids or for hydrogenating free fatty acids or fatty acid esters to fatty alcohols comprising catalyzing with a mixed oxide according to claim 7.

16. Catalyst, containing the mixed oxide containing copper and chromium according to claim 7.

17. Catalyst, characterized in that it is present in the form of a bulk catalyst, shell catalyst or as extrudate.

18. Catalyst according to claim 16, characterized in that it further contains a binder.
